(19) United States
(12) Patent Application Publication
(10) Pub. No.: US 2023/0263509 A1
(43) Pub. Date: Aug. 24, 2023

(54) CAPSULE FOR FLUID SAMPLING AND DRUG DELIVERY

(71) Applicants: ANKON TECHNOLOGIES CO., LTD, Wuhan (CN); ANX IP HOLDING PTE. LTD., Singapore (SG)

(72) Inventors: Tianyi Yangdai, Wuhan (CN); Yuhui BAO, WUHAN (CN); Hangyu PENG, WUHAN (CN); Fanhua MING, Wuhan (CN)

(73) Assignees: ANKON TECHNOLOGIES CO., LTD, Wuhan (CN); ANX IP HOLDING PTE. LTD., Singapore (SG)

(21) Appl. No.: 17/922,496

(22) PCT Filed: Apr. 27, 2021
(86) PCT No.: PCT/CN2021/090280

§ 371 Date: Oct. 31, 2022

(30) Foreign Application Priority Data
Apr. 30, 2020 (CN) 202010360917.8

(51) Int. Cl. A61B 10/00; A61B 1/04; A61B 1/045

(52) U.S. Cl. CPC A61B 10/0045; A61B 1/041; A61B 1/045; A61B 2010/0061

(57) ABSTRACT

The present invention provides a capsule for fluid sampling and drug delivery comprising: an enclosure, comprising a first enclosure, a second enclosure, at least a portion of the first enclosure comprising a support body having a plurality of first openings, a liquid-proof air-permeable membrane located at the first openings; a sampling/drug delivery assembly, comprising a storage chamber at least partially enclosed by the liquid-proof air-permeable membrane, a temporary storage chamber located in the enclosure, a first one-way element connecting the temporary storage chamber to outside of the capsule, a second one-way element connecting the temporary storage chamber to the storage chamber, and a pump changing pressure in the temporary storage chamber, wherein the first one-way element and the second one-way element have same direction of access; and a control module, comprising a microprocessor in communication with the pump. The capsule can realize multiple times of sampling and drug delivery.

### CROSS-REFERENCE OF RELATED APPLICATIONS

The application is a National Phase Application of PCT International Application No. PCT/CN2021/090280, International Filing Date Apr. 27, 2021, published Nov. 4, 2021 as International Publication Number WO2021/218972A1, which claims priority from Chinese Patent Application No. 202010360917.8, filed Apr. 30, 2020, all of which are incorporated herein by reference in their entirety.

### FIELD OF INVENTION

The present invention relates to a medical device, in particular to a capsule capable of sampling fluid and delivering drugs for multiple times.

### BACKGROUND

At present, there are many system designs for gastrointestinal fluid sampling or drug delivery. However, existing device for gastrointestinal sample collection or drug delivery can only perform a single action. For fluid sampling, if a single sampling fails, for example, air is collected, the device is ineffective and a new device is required.

In addition, existing gastrointestinal fluid sampling device is passive in sampling and its sampling rate and volume of sample that it collects are difficult to estimate. If sampling time is too long, sampling area may change greatly, leading to a low accuracy of positioning. If the sampling time is too short, the collected volume of sample may be insufficient.

To solve the problem, it is necessary to provide an improved capsule for fluid sampling and drug delivery.

### SUMMARY OF THE INVENTION

The present invention provides a capsule capable of sampling fluid and delivering drugs for multiple times.

To achieve the above-mentioned purpose of the present invention, the present invention uses the following technical solutions:

a capsule for fluid sampling and drug delivery comprising:

an enclosure, comprising a first enclosure and a second enclosure, at least a portion of the first enclosure comprising a support body having a plurality of first openings, a liquid-proof air-permeable membrane located within the support body and at the first openings;

a sampling/drug delivery assembly, comprising a storage chamber at least partially enclosed by the liquid-proof air-permeable membrane, a temporary storage chamber located in the enclosure, a first one-way element connecting the temporary storage chamber to outside of the capsule, a second one-way element connecting the temporary storage chamber to the storage chamber, and a pump changing pressure in the temporary storage chamber, wherein the first one-way element and the second one-way element have same direction of access; and

a control module, comprising a microprocessor in communication with the pump.

Further, an area of the first openings is between 0.8 mm² and 3 mm².

Further, the first one-way element and the second one-way element are respectively selected from a one-way valve or a one-way membrane.

Further, the pump comprises a pneumatic pump that repeatedly pumps air out of and into the temporary storage chamber, and an air storage structure that communicates with the pneumatic pump;

alternatively, the pump comprises a diaphragm that communicates with the temporary storage chamber, and a deformation element that repeatedly deforms at least part of the diaphragm to change the pressure in the temporary storage chamber.

Further, the deformation element may be at least one of a piezoelectric micropump based on a piezoelectric material, a bimetallic micropump based on a bimetal with dual thermal deformation coefficients, and a micropump based on a shape memory alloy.

Further, suction force and thrust force of the pump are not less than pressure of forward conduction of the first one-way element and the second one-way element, and the suction force and the thrust force of the pump are not greater than pressure of reverse withstand of the first one-way element and the second one-way element.

Further, pressure threshold of forward conduction of the first one-way element and the second one-way element is 5 kPa-15 kPa, and pressure threshold of reverse withstand is 50 kPa 100 kPa; and the suction force of the pump is between 20 kPa-30 kPa and the thrust force of the pump is between 30 kPa 50 kPa.

Further, the capsule further comprises a partition wall disposed in the enclosure and located at the connection between the first enclosure and the second enclosure, and a spacing wall arranged at a side of the partition wall facing the second enclosure, wherein the partition wall and the first enclosure form the storage chamber, and the partition wall, the spacing wall and a structure between the two jointly form the temporary storage chamber; and wherein the second enclosure comprises a passage communicating with the temporary storage chamber, and the first one-way element is arranged in the passage; and wherein the partition wall comprises a second opening in which the second one-way element is fixed.

Further, at least part of the first openings is arranged on a portion of the first enclosure adjacent to the partition wall, and the passage is disposed on a portion of the second enclosure adjacent to the partition wall.

Further, the passage comprises a plurality of sample holes disposed on the second enclosure and a sample chamber communicating with the plurality of sample holes. The first one-way element connects the sample chamber to the temporary storage chamber.

Further, an aperture diameter of each of the sample holes is smaller than an aperture diameter of the first one-way element in communication with the sample chamber, and/or the sample chamber comprises a filter structure.

According to all aspects of the present invention, the capsule for fluid sampling and drug delivery can realize multiple times of sampling and drug delivery by means of the cooperation of the first one-way element, the second one-way element, the liquid-proof air-permeable membrane and the pump.

### BRIEF DESCRIPTION OF THE DRAWINGS

FIG. 1 is a cross-sectional view of a capsule for fluid sampling and drug delivery cut along an axis direction according to a preferred embodiment of the present invention.

FIG. 2 is a schematic view of the capsule shown in FIG. 1 in an operating state where gastrointestinal fluid flows into a temporary storage chamber of the capsule.

FIG. 3 is a schematic view of the capsule shown in FIG. 1 in an operating state where the gastrointestinal fluid flows from the temporary storage chamber into a storage chamber of the capsule.

FIG. 4 is a cross-sectional view of the capsule for fluid sampling and drug delivery cut along the axis direction according to another preferred embodiment of the present invention.

FIG. 5 is a planar view of a first enclosure of the capsule of FIGS. 1 to 4.

FIG. 6 is a sectional view of the first enclosure of FIGS. 1 to 4.

### DETAILED DESCRIPTION

The present invention will be described in detail below with reference to accompanying drawings and preferred embodiments. However, the embodiments are not intended to limit the present invention, and structural, method, or functional changes made by those skilled in the art in accordance with the embodiments are included in the scope of the present invention.

In the figures of the present invention, some dimensions of a structure or portion may be exaggerated relative to other structures or portions for ease of illustration, and thus, are merely used to illustrate basic structure of the present invention.

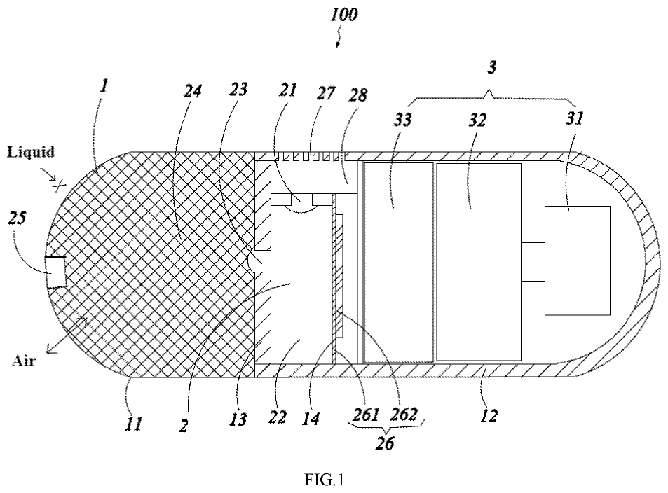

Referring to FIGS. 1 to 6, showing a preferred embodiment of a capsule for fluid sampling and drug delivery 100 according to the present invention, which comprises an enclosure 1, a sampling/drug delivery assembly 2, and a control module 3. The control module 3 comprises a microprocessor in communication with at least a portion of structure of the sampling/drug delivery assembly 2 to control an operating state of the capsule.

The enclosure 1 is biocompatible and is not corroded by gastrointestinal fluid, and can be transparent or opaque as needed. In particular, the enclosure 1 comprises a first enclosure 11 and a second enclosure 12.

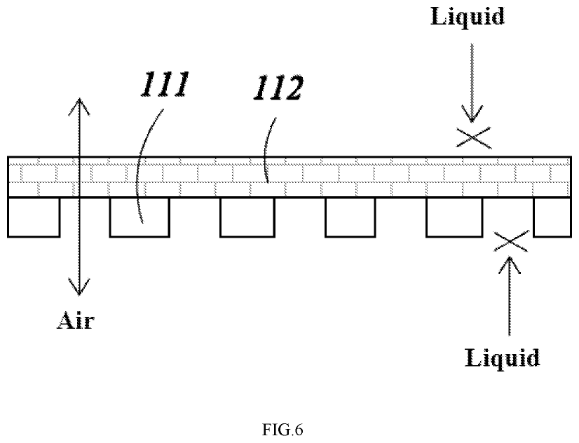

Referring to FIGS. 5 and 6, at least a portion of the first enclosure 11 comprises a support body 111 having a plurality of first openings, and a liquid-proof air-permeable membrane 112 located within the support body 111 and at the first openings. Preferably, all of the first enclosure 11 comprises the support body 111 and the liquid-proof air-permeable membrane 112.

Specifically, the density of the first openings provided on the support body 111 is not limited, the support body 111 may only comprises a few of the first openings, and the support body 111 may also be a grid structure with a large number of the first openings, and the first openings may also be called grid holes at this point. The first openings are not limited in shape, and may be a circular hole, a square hole or other irregular holes. For example, when the support body 111 is of the grid structure, an aperture diameter of the first openings or grid holes is between 10 mesh and 18 mesh, or the aperture diameter is 1 mm to 2 mm, or an area of the first openings or grid holes is 0.8 mm² to 3 mm².

The liquid-proof air-permeable membrane 112 has a good permeability to air, and has a good blocking performance to liquid, so that air can freely enter and exit the enclosure 1 through the first enclosure 11, while the gastrointestinal fluid cannot freely pass through. In a specific embodiment, the waterproof rating of the liquid-proof air-permeable membrane 112 is IPX8.

The sampling/drug delivery assembly 2 comprises a storage chamber 24 at least partially enclosed by the liquid-proof air-permeable membrane 112, a temporary storage chamber 22 located in the enclosure 1, a first one-way element 21 connecting the temporary storage chamber 22 to outside of the capsule 100, a second one-way element 23 connecting the temporary storage chamber 22 to the storage chamber 24, and a pump 26 changing pressure in the temporary storage chamber 22, wherein the first one-way element 21 and the second one-way element 23 have same direction of access. The storage chamber 24 may communicate with the outside of the capsule 100 through the liquid-proof air-permeable membrane 112. The pump 26 is an electronically controlled element, communicatively connected to the microprocessor.

It will be understood by those skilled in the art that “the first one-way element 21 and the second one-way element 23 have same direction of access” means that the direction of flow for sampling samples or drugs is the same. Two situations are included. One is that the first one-way element 21 leads to the temporary storage chamber 22, and the second one-way element 23 leads to the storage chamber 24, and at this point, sampling function can be realized. Alternatively, another is that the second one-way element 23 leads to the temporary storage chamber 22, and the first one-way element 21 leads to the outside of the capsule 100, and at this point, drug delivery function can be realized.

The capsule for fluid sampling and drug delivery 100 integrates the sampling function and the drug delivery function, and can be used as a sampling capsule and a drug delivery capsule. When it is used as the sampling capsule for sampling, the storage chamber 24 is empty for storing samples collected at specific sites of gastrointestinal tract. When it is used as the drug delivery capsule for delivery of drugs, the storage chamber 24 is filled with drugs to be released, mostly liquid drug, and the drugs are released outward when the capsule 100 reaches a specific site of the gastrointestinal tract.

Specifically, in the embodiment as shown in FIG. 1, the capsule 100 further comprises a partition wall 13 disposed in the enclosure 1 and located at the connection between the first enclosure 11 and the second enclosure 12, and the partition wall 13 and the first enclosure 11 form the storage chamber 24. The partition wall 13 is located at the connection of the first enclosure 11 and the second enclosure 12 and divides the interior of the first enclosure 11 and the second enclosure 12 into two spaces. Specifically, the partition wall 13 is located at an end of the first enclosure 11 facing the second enclosure 12, or, the partition wall 13 is located between the first enclosure 11 and the second enclosure 12. Alternatively, as shown in FIG. 1, the partition wall 13 is located at an end of the second enclosure 12 facing the first enclosure 11.

The capsule 100 further comprises a spacing wall 14 arranged at a side of the partition wall 13 facing the second enclosure 12. The partition wall 13, the partition wall 14 and a structure between the two jointly form the temporary storage chamber 22. In an embodiment, the temporary storage chamber 22 is enclosed jointly by the partition wall 13, the spacing wall 14 and the second enclosure 12 between the two, or the structure attached to the second enclosure 12.

The first one-way element 21 is fixed to the second enclosure 12 and connects the temporary storage chamber 22 to the outside of the enclosure 1. The second one-way element 23 is fixed to the partition wall 13 and connects the temporary storage chamber 22 to the storage chamber 24. Preferably, the second one-way element 23 is fixed to the middle of the partition wall 13, allowing sample/drug to flow through smoothly.

The first one-way element 21 and the second one-way element 23 are respectively selected from a one-way valve or a one-way membrane. The one-way valve only allows the gastrointestinal fluid and air to pass through in a single direction, and can bear a pressure of 50 kPa˜100 kPa in the reverse direction without infiltration. The one-way valve is installed in such a way that an opening is cut in a corresponding position and the one-way valve is sealed corresponding to the opening, for example, the one-way valve is sealed and disposed in the opening. Likewise, the one-way membrane only allows fluid and air to pass through in a single direction and is installed in such a way that an opening is cut in a corresponding position, and the one-way membrane covers the opening.

Specifically, a passage is provided on the second enclosure 12 between the partition wall 13 and the spacing wall 14, and the first one-way element 21 is provided on the passage to control the sample or drug to pass through the passage in the single direction. The partition wall 13 has a second opening, and the second one-way element 23 is fixed corresponding to the second opening, for example, the second one-way element 23 is fixed in the second opening.

Preferably, at least part of the first openings is arranged on a portion of the first enclosure 11 adjacent to the partition wall 13, and the passage is disposed on a portion of the second enclosure 12 adjacent to the partition wall 13. Then, the first opening is close to the passage, and there is only one partition wall 13 in the middle, which can maintain the pressure difference of each cavity of the system within an acceptable range, and cannot cause the pump 26 to fail due to excessive load.

Further, the passage comprises a plurality of sample holes 27 disposed on the enclosure 1 and a sample chamber 28 communicating with the plurality of sample holes 27. The first one-way element 21 connects the sample chamber 28 to the temporary storage chamber 22.

When the first one-way element 21 leads to the temporary storage chamber 22 and the second one-way element 23 leads to the storage chamber 24, the sample holes 27 are sampling holes, which can prevent blocking.

During sampling, the air and fluid in the gastrointestinal tract enter the sample chamber 28 through the sample holes 27 and converge into the temporary storage chamber 22. Even if some of the sample holes 27 are blocked, rest of them remain in good condition and do not affect sampling. In addition, the gastrointestinal fluid entering through each sample hole 27 can be mixed and buffered through the sample chamber 28 to ensure uniform and smooth sampling.

Further, an aperture diameter of each of the sample holes 27 is smaller than an aperture diameter of the first one-way element 21 communicating with the sample chamber 28, so that substances that can enter the sample chamber 28 through the sample holes 27 do not block the temporary storage chamber 22.

Alternatively, the sample chamber 28 comprises a filter structure (not shown in FIGS), such as, but not limited to, a filter screen, to avoid food residues blocking the temporary storage chamber 22. The filter structure of the sample chamber 28 can be referred to a filter structure designed in Chinese Patent Application No. 201811330328.4.

Alternatively, while the aperture diameter of each of the sample holes 27 is smaller than the aperture diameter of the first one-way element 21 communicating with the sample chamber 28, a filter structure may be provided in the sample chamber 28 to achieve a double anti-blocking effect.

The plurality of sample holes 27 are distributed with spacing along lengthwise direction or circumferential direction of the capsule 100 to ensure smooth sampling/drug delivery.

Alternatively, the second one-way element 23 leads to the temporary storage chamber 22 and the first one-way element 21 to the outside of the capsule 100. Based on such direction of access, when the drug is delivered, the drug enters the temporary storage chamber 22 from the storage chamber 24, and then enters the sample chamber 28 from the temporary storage chamber 22 where it is fully mixed and is released to the gastrointestinal tract through the plurality of sample holes 27, which expands the range of drug delivery.

In an embodiment, the sample holes 27 are arranged close to the partition wall 13, and the entire first enclosure 11 comprises the support body 111 and the liquid-proof air-permeable membrane 112. The first opening/the liquid-proof air-permeable membrane 112 is very close to the sample holes 27, with only the partition wall 13 set between the two, which can maintain pressure difference of each cavity within an acceptable range, and cannot cause the pump 26 to fail due to excessive load.

Pressure in the storage chamber 24 is set to P0, and pressure in the temporary storage chamber 22 is set to P1, and external pressure of the capsule 100 is set to P2. The first enclosure 11 has a plurality of first openings, and the liquid-proof air-permeable membrane 112 has good air permeability, so that the pressure inside and outside the liquid-proof air-permeable membrane 112 can reach equilibrium in a small period of time, i.e., the pressure in the storage chamber 24 is equal to the external pressure. Since the temporary storage chamber 22 is connected to the outside through the first one-way element 21 and the second one-way element 23, the pressure P1 in the temporary storage chamber 22 may be different from the pressure P0. According to the performance of the one-way valve, when the system of the capsule reaches equilibrium, (A) no air or liquid flows into the temporary storage chamber 22; (B) no air or liquid flows into the storage chamber 24 from the temporary storage chamber 22. At this point, it is stated that:

P0−P1≤ΔP; (A)

P1−P0≤ΔP. (B)

In combination with the above conditions, |P1−P0|≤ΔP. where ΔP is a pressure threshold of forward conduction of the first one-way element 21 and the second one-way element 23.

Specifically, if the first enclosure 11 outside the entire storage chamber 24 is of a porous structure such as a grid structure, with a liquid-proof air-permeable membrane 112 covered therein, and the sample holes 27 are spaced from the first enclosure 11 by only one partition wall 13, preferably, the thickness of the partitioned partition wall 13 is about 1 mm. Thus, the sample holes 27 and some holes in the first enclosure 11 are substantially in the same space. If ambient pressure in local space of the intestine changes due to peristalsis or the like, the pressure in the storage chamber 24 changes synchronously.

If the external pressure increases, that is, P2>P0 and P2>P1+ΔP, the external air and liquid can enter the temporary storage chamber 22 through the first one-way element 21, and the pressure of the temporary storage chamber 22 can gradually increase. When the pressure in the temporary storage chamber 22 reaches P2-ΔP, the external pressure is insufficient to push away the first one-way element 21, and the air and liquid stop flowing inward. At this time, the internal air and liquid cannot push away the second one-way element 23 to flow into the storage chamber 24.

If the external pressure decreases, that is, P2<P0 and P2+ΔP<P1, the air and liquid in the temporary storage chamber 22 can enter the storage chamber 24 until the pressure in the temporary storage chamber 22 decreases to P2+ΔP. At this time, the external air and liquid cannot enter the temporary storage chamber 22 through the first one-way element 21.

To sum up, when the ambient pressure changes due to intestinal peristalsis after the pump 26 is turned on, the pressure in the temporary storage chamber 22 can always be maintained between ±ΔP of the ambient pressure. At the same time, the pressure in the storage chamber 24 can be maintained consistent with the ambient pressure outside the liquid-proof air-permeable membrane 112 (sample holes 27). Since both suction force and thrust force of the pump 26 are higher than ΔP, the throughput of the pump 26 cannot fail, i.e., the pump 26 cannot be unable to operate due to excessive load.

The pump 26 is used to change the pressure in the temporary storage chamber 22, in conjunction with the passage of the first one-way element 21 and the second one-way element 23, and the liquid-proof air-permeable membrane 112 to complete sampling or drug delivery.

In one embodiment, as shown in FIGS. 1 to 3, the first one-way element 21 leads to the temporary storage chamber 22, and the second one-way element 23 leads to the storage chamber 24. The flow direction of the gastrointestinal fluid is: flowing in sequence through the first one-way element 21, the temporary storage chamber 22, the second one-way element 23 and the storage chamber 24, and the air doped in the gastrointestinal fluid can be discharged to the outside of the enclosure 1 through the liquid-proof air-permeable membrane 112 in this process.

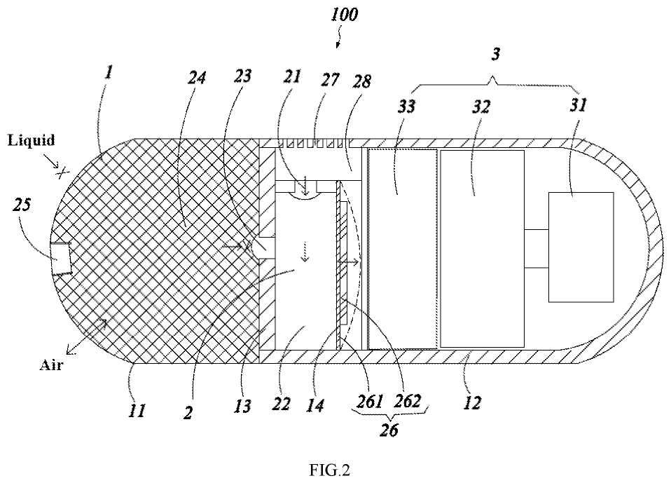

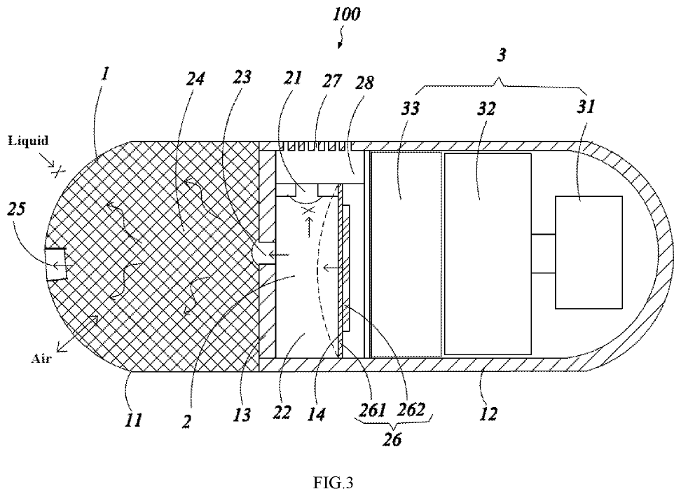

The specific sampling process is: as shown in FIG. 2, the pump 26 reduces the pressure of the temporary storage chamber 22, and the gastrointestinal fluid enters the temporary storage chamber 22 through the first one-way element 21 under the action of the internal and external pressure difference; then, as shown in FIG. 3, the pump 26 increases the pressure in the temporary storage chamber 22, and the gastrointestinal fluid enters the storage chamber 24 through the second one-way element 23. Then, the sampling is completed.

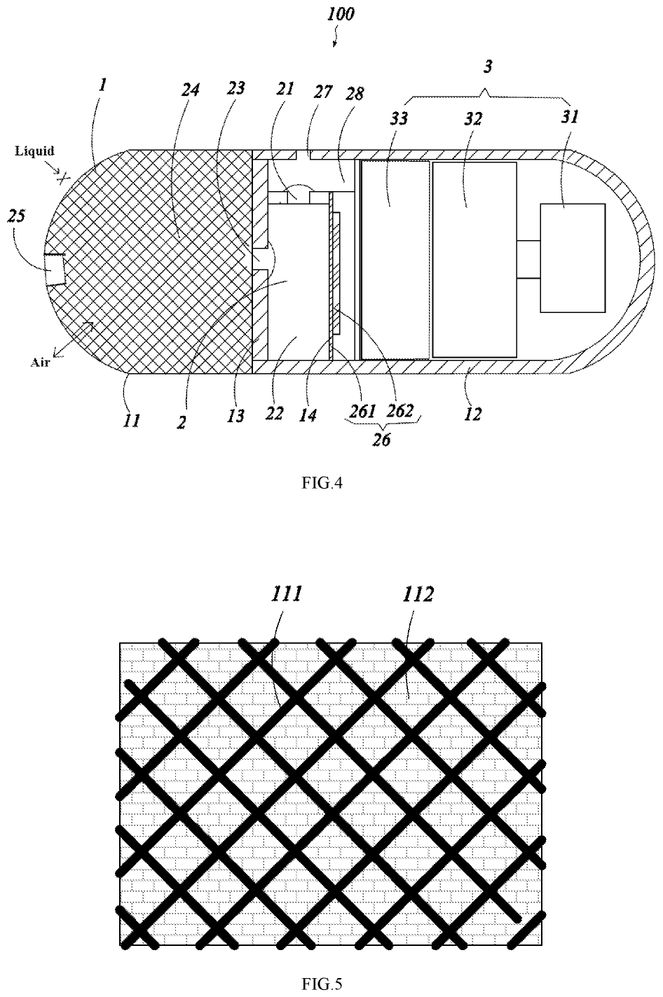

In another embodiment, as shown in FIG. 4, the second one-way element 23 leads to the temporary storage chamber 22, and the first one-way element 21 leads to the outside of the capsule 100. During the operation of the pump 26, part of the air in the gastrointestinal tract flows through the liquid-proof air-permeable membrane 112 into the storage chamber 24 to increase the pressure thereof, so that the drug stored therein flows into the temporary storage chamber 22 through the second one-way element 23, and then flows out into the gastrointestinal tract through the first one-way element 21.

The specific drug delivery process is: the pump 26 reduces the pressure of the temporary storage chamber 22, the air/drug in the storage chamber 24 flows into the temporary storage chamber 22 through the second one-way element 23 to reduce the pressure of the storage chamber 24, and the air in the gastrointestinal tract flows into the storage chamber 24 through the liquid-proof air-permeable membrane 112 under the action of the internal and external pressure difference, pushing the drug to flow through the second one-way element 23 into the temporary storage chamber 22; then, the pump 26 increases the pressure in the temporary storage chamber 22, so that the drug is discharged out of the capsule 100 through the first one-way element 21.

In order to ensure the one-way passage of the gastrointestinal fluid or drug, the suction force and thrust force of the pump 26 are not less than pressure of forward conduction of the first one-way element 21 and the second one-way element 23, and the suction force and thrust force of the pump 26 are not greater than pressure of reverse withstand of the first one-way element 21 and the second one-way element 23.

In an embodiment, the pressure threshold of forward conduction of the first one-way element 21 and the second one-way element 23 is 5 kPa-15 kPa, and pressure threshold of reverse withstand is 50 kPa 100 kPa. The suction force of the pump 26 is between 20 kPa-30 kPa and the thrust force is between 30 kPa 50 kPa.

Specifically, in one embodiment, the pump 26 comprises a diaphragm 261 that communicates with the temporary storage chamber 22, and a deformation element 262 that repeatedly deforms at least part of the diaphragm 261 to change the pressure in the temporary storage chamber 22. Specifically, the diaphragm 261 is disposed on the spacing wall 14, or the spacing wall 14 in its entirety is the diaphragm 261. When the diaphragm 261 is deformed, the volume of the temporary storage chamber 22 changes, and thus the pressure of the temporary storage chamber 22 changes.

In an embodiment, the deformation element 262 is a piezoelectric micropump based on piezoelectric material. When a voltage is applied to the piezoelectric micropump, the piezoelectric material can bend, driving the diaphragm 261 to bend to increase or decrease the pressure in the temporary storage chamber 22.

Take sampling as an example. Referring to FIG. 2 and FIG. 3, when a voltage is applied to the piezoelectric micropump, the piezoelectric material bends away from the storage chamber 24, driving the diaphragm 261 to bend away from the storage chamber 24 to increase the volume of the temporary storage chamber 22 and reduce the pressure of the temporary storage chamber 22, and the gastrointestinal fluid flows into the temporary storage chamber 22 through the first one-way element 21 under the action of the internal and external pressure difference. When the applied voltage is removed, the piezoelectric material restores to its original shape and drives the diaphragm 261 to restores to its original shape, or when a reverse voltage is applied, the piezoelectric material bends toward the storage chamber 24 and drives the diaphragm 261 to bend toward the storage chamber 24 to reduce the volume of the temporary storage chamber 22, and the pressure in the temporary storage chamber 22 increases, so that the gastrointestinal fluid flows into the storage chamber 24 through the second one-way element 23 to complete sampling.

When the sample is collected, after air enters the storage chamber 24, the air is discharged from the capsule 100 through the liquid-proof air-permeable membrane 112, and the above operation is repeated to complete the sample injection again. After multiple sampling, the collected liquid can be stored, and the sucked air can be automatically discharged to ensure the collection of liquid samples such as gastrointestinal fluid, thus avoiding the problem of insufficient sampling volume or failure due to the sucked air occupying the space of the storage chamber 24.

Alternatively, the deformation element 262 is a bimetallic micropump based on a bimetal with dual deformation coefficients, and the bimetallic micropump is composed of two different metal diaphragms that are relatively fixed. Since the deformation coefficients of the two metal diaphragms are different, when they are heated by a heating element, such as a resistor, the two metal diaphragms are deformed to different degrees, making the bimetallic micropump deformed, and thus deforming the diaphragm 261, and then changing the pressure of the temporary storage chamber 22.

As shown in FIG. 2, during heating, the bimetallic micropump bends away from the storage chamber 24, thereby causing the diaphragm 261 to bend away from the storage chamber 24, reducing the pressure of the temporary storage chamber 22. After the heating is stopped and the temperature is restored, the shape of the bimetallic micropump 26 is restored, the shape of the diaphragm 261 is restored, and the pressure in the temporary storage chamber 22 is increased.

In this embodiment, the principle and process of sampling based on pressure change are the same as those of the above embodiments, and will not be repeated here.

Alternatively, the deformation element 262 is a micropump based on a shape memory alloy. The micropump based on the shape memory alloy is made of the shape memory alloy. When temperature of the shape memory alloy is raised to a certain value by using a heating element, the shape memory alloy can deform and cause the diaphragm 261 to deform, thereby changing the pressure of the temporary storage chamber 22.

As shown in FIG. 2, during heating, the micropump based on the shape memory alloy bends away from the storage chamber 24, thereby causing the diaphragm 261 to bend away from the storage chamber 24, reducing the pressure of the temporary storage chamber 22. After the heating is stopped and the temperature is restored, the shape of the micropump based on the shape memory alloy is restored, the shape of the diaphragm 261 is restored, and the pressure in the temporary storage chamber 22 is increased.

In this embodiment, the principle and process of sampling based on pressure change are the same as those of the above embodiments, and will not be repeated here.

When a signal capable of deforming the deformation element 262 is periodically applied, such as applying voltage and heating, so that the deformation element 262 repeatedly switches between deformation and restoration, the pressure of the temporary storage chamber 22 can repeatedly decrease and then increase, and sampling can be performed multiple times to avoid various disadvantages of single sampling.

Alternatively, in other embodiments, the pump 26 comprises a pneumatic pump 26 that can repeatedly pump air out of and into the temporary storage chamber 22, and an air storage structure that communicates with the pneumatic pump 26. The pneumatic pump 26 changes air pressure of the temporary storage chamber 22 by pumping the air in the temporary storage chamber 22 into the air storage structure or pumping the air in the air storage structure into the temporary storage chamber 22, thereby controlling the flow of the sample.

The volume of the air storage structure is set such that when the air flows in the temporary storage chamber 22 and the air storage structure, a sufficient pressure difference can be generated to meet the requirements of sampling or drug delivery.

Further, the sampling/drug delivery assembly 2 further comprises a third opening disposed on the first enclosure 11 and a rubber stopper 25 located in the third opening. For example, the rubber stopper 25 is installed in interference fit with the third opening, which not only facilitates injection of drugs into the storage chamber 24, but also facilitates recovery of samples by piercing the rubber stopper 25 by a syringe or the like after sampling.

In general, fluid sampling or drug delivery may be performed when the capsule 100 is at a site of the gastrointestinal tract to be examined, or reaches a site of the gastrointestinal tract with lesions.

The control module 3 further comprises a sensor 31 for collecting physiological parameters and/or image information in the gastrointestinal tract, and the sensor 31 communicates with the microprocessor. The sensor 31 is at least an image sensor, or a pH sensor, or an ultrasonic sensor. When the sensor 31 comprises an image sensor, part of the enclosure 1 is transparent, and when the sensor 31 comprises a pH sensor, the enclosure 1 has a window. The specific method of determining which site of the gastrointestinal tract the capsule 100 is in, based on the images and pH value obtained by the sensor 31, can be any method in the prior art, and will not be repeated herein.

In addition to the sensor 31, the control module 3 can further comprise a storage module for storing physiological parameters and image information in different regions of the gastrointestinal tract, where the storage module communicates with the microprocessor. The parameters and image information comprise normal physiological parameters or normal image information, and physiological parameters or image information in case of possible lesions. When the sensor 31 collects physiological parameters and/or image information in the gastrointestinal tract, the microprocessor compares the above information with the information in the storage module to determine whether the capsule 100 reaches a position at which the sample is to be collected or drug is to be released.

Alternatively, in addition to the sensor 31, the control module 3 further comprises a wireless transmission module for communicating with an external processing terminal. When the sensor 31 collects the physiological parameters and/or the image information in the gastrointestinal tract, the information is transmitted to the external processing terminal, and the external processing terminal analyzes the information and determines whether the capsule 100 has reached the position at which the sample is to be collected or drug is to be released.

In addition, the control module 3 further comprises a battery 32 for supplying power to other components, and the microprocessor and the wireless transmission module are generally integrated on a same circuit board 33.

The capsule 100 of the present invention can communicate with the external processing terminal through the control module 3, and the control module communicates with the external processing terminal by any of the methods in the prior art, it will not be repeated herein.

To sum up, the capsule for fluid sampling and drug delivery 100 of the present invention can realize multiple times of sampling and drug delivery by means of the cooperation of the first one-way element 21, the second one-way element 23, the liquid-proof air-permeable membrane 112 and the pump 26.

It should be understood that, although the specification is described in terms of embodiments, not every embodiment merely comprises an independent technical solution, and the specification is described in this manner only for clarity. Those skilled in the art should have the specification as a whole, and the technical solutions in each embodiment may also be combined as appropriate to form other embodiments that can be understood by those skilled in the art.

The series of detailed descriptions set forth above are only specific descriptions of feasible embodiments of the present invention and are not intended to limit the scope of protection of the present invention. Any equivalent embodiments or modifications made without departing from the spirit of the art of the present invention shall be included within the scope of protection of the present invention.

1. A capsule for fluid sampling and drug delivery comprising:
an enclosure, comprising a first enclosure and a second enclosure, at least a portion of the first enclosure comprising a support body having a plurality of first openings, a liquid-proof air-permeable membrane located within the support body and at the first openings;
a sampling/drug delivery assembly, comprising a storage chamber at least partially enclosed by the liquid-proof air-permeable membrane, a temporary storage chamber located in the enclosure, a first one-way element connecting the temporary storage chamber to outside of the capsule, a second one-way element connecting the temporary storage chamber to the storage chamber, and a pump changing pressure in the temporary storage chamber, wherein the first one-way element and the second one-way element have same direction of access; and
a control module comprising a microprocessor in communication with the pump.

2. The capsule of claim 1, wherein an area of the first openings is between 0.8 mm² and 3 mm².

3. The capsule of claim 1, wherein the first one-way element and the second one-way element are respectively selected from a one-way valve or a one-way membrane.

4. The capsule of claim 1, wherein the pump comprises a pneumatic pump that repeatedly pumps air out of and into the temporary storage chamber, and an air storage structure that communicates with the pneumatic pump;
or, the pump comprises a diaphragm that communicates with the temporary storage chamber, and a deformation element that repeatedly deforms at least part of the diaphragm to change the pressure in the temporary storage chamber.

5. The capsule of claim 4, wherein the deformation element is at least one of a piezoelectric micropump based on a piezoelectric material, a bimetallic micropump based on a bimetal with dual thermal deformation coefficients, and a micropump based on a shape memory alloy.

6. The capsule of claim 1, wherein suction force and thrust force of the pump are not less than pressure of forward conduction of the first one-way element and the second one-way element, and the suction force and the thrust force of the pump are not greater than pressure of reverse withstand of the first one-way element and the second one-way element.

7. The capsule of claim 6, wherein pressure threshold of forward conduction of the first one-way element and the second one-way element is 5 kPa-15 kPa, and pressure threshold of reverse withstand is 50 kPa 100 kPa; and the suction force of the pump is between 20 kPa-30 kPa and the thrust force of the pump is between 30 kPa 50 kPa.

8. The capsule of claim 1, further comprising:
a partition wall disposed in the enclosure and located at the connection between the first enclosure and the second enclosure, and a spacing wall arranged at a side of the partition wall facing the second enclosure, wherein the partition wall and the first enclosure form the storage chamber, and the partition wall, the spacing wall and a structure between the two jointly form the temporary storage chamber; and wherein the second enclosure comprises a passage communicating with the temporary storage chamber, and the first one-way element is arranged in the passage; and wherein the partition wall comprises a second opening in which the second one-way element is fixed.

9. The capsule of claim 8, wherein at least part of the first openings is arranged on a portion of the first enclosure adjacent to the partition wall, and the passage is disposed on a portion of the second enclosure adjacent to the partition wall.

10. The capsule of claim 8, wherein the passage comprises a plurality of sample holes disposed on the second enclosure and a sample chamber communicating with the plurality of sample holes; and wherein the first one-way element connects the sample chamber to the temporary storage chamber.

11. The capsule of claim 10, wherein an aperture diameter of each of the sample holes is smaller than an aperture diameter of the first one-way element in communication with the sample chamber, and/or the sample chamber comprises a filter structure.
